(19) United States
(12) Patent Application Publication
(10) Pub. No.: US 2009/0324517 A1
(43) Pub. Date: Dec. 31, 2009

(54) HALITOSIS TREATMENT

(76) Inventor: Dan Kline, Chandler, AZ (US)

Correspondence Address: SCHMEISER OLSEN & WATTS, 18 E UNIVERSITY DRIVE, SUITE # 101, MESA, AZ, 85201, US

(21) Appl. No.: 12/165,316

(22) Filed: Jun. 30, 2008

(51) Int. Cl. A61K 8/22; A61Q 11/00; A61P 31/02

(52) U.S. Cl. 424/53

(57) ABSTRACT

A method of oral treatment to eliminate odors, particularly halitosis, using two or more oral treatment compositions. A four step method in a sequential order may be followed using compositions including hydrogen peroxide, metal ions, bactericidal agents and essential oils. Gargling increases the effectiveness of the treatment. Good breath is maintained with a two step maintenance treatment.

### BACKGROUND OF THE INVENTION

1. Technical Field

This invention relates generally to a method for oral treatment, particularly to a method for treatment of halitosis and more particularly to a method for treating halitosis with gargling in a predetermined manner.

2. State of the Art

Contrary to popular thought, halitosis does not come from the stomach. There is a physical closure between the esophagus and stomach. Nothing is transmitted through this closure from the stomach to the mouth unless you burp or regurgitate.

Food breath is not halitosis. Garlic, onions, spices and alcohol create characteristic odors, but if these substances are avoided the odor is not present. This is easily controlled and is not considered halitosis. Garlic breath, for instance, remains long after garlic residues have been cleared from the mouth, for several hours, because the odor comes from the lungs. As garlic is digested several byproducts are formed. Only one, however, is not quickly further metabolized by the gut and liver into non-odiferous compounds. Allyl methyl sulfide (AMS) has the familiar odor of garlic breath and is eliminated from the body by the excretory functions of the lungs, skin and kidneys. Studies that compare fresh garlic with various garlic preparations are commonly done at 30 hours after ingestion and are measured in mouth air. There is presently no well accepted or proven remedy for this type of breath.

Eighty-five to ninety percent of halitosis originates in the mouth. Numerous studies support this assertion. Of the remaining sites of origin, the nose and sinuses contribute five to ten percent, the tonsils three percent and all other sources account for one percent.

Halitosis odor is formed by bacterial byproducts during the metabolism primarily of certain proteins. This results in sulfur compounds, for example, hydrogen sulfide, which has the smell of rotten eggs, which are distinctive and objectionable, as well as other compounds that contribute to the smell.

The causative bacteria reside mainly on the posterior of the tongue, especially the extreme posterior, which cannot be seen when looking in the mirror with the tongue out as far as possible, and the oral pharynx, particularly when tonsils are present. Mechanical cleansing of the back of the tongue by brushing or scraping creates temporary reductions in halitosis. However, brushing and flossing do not relieve morning breath as well as eating breakfast. It is believed that eating affects the rear of the tongue and throat, while brushing and flossing do not. The vast majority of halitosis is caused by odors released from bacteria on the back of the tongue.

Accordingly, there is a need in the field of oral treatment for a treatment regimen to eliminate halitosis odor. Furthermore, there is a need for the treatment regimen to eliminate halitosis odor to reach the back of the tongue.

### DISCLOSURE OF THE INVENTION

The present invention relates to an oral treatment method comprising applying at least two, particularly at least three oral treatment compositions. The oral treatment method includes a treatment regimen in a predetermined order.

An aspect of the present invention includes a method for providing oral treatment comprising the steps of applying at least two compositions to a human oral cavity selected from the group consisting of a first composition comprising hydrogen peroxide; a second composition comprising a metal selected from the group consisting of copper, tin and/or zinc; a third composition comprising a bactericidal agent; and/or a fourth composition comprising essential oils.

Another aspect of the present invention includes a method for providing oral treatment comprising the steps of applying a first composition comprising hydrogen peroxide to a human oral cavity; applying a second composition comprising a metal selected from the group consisting of copper, tin and/or zinc after said first composition; applying a third composition comprising chlorhexidine after said second composition; and applying a fourth composition comprising essential oils after said third composition.

Other aspects of the present invention may include a method for providing oral treatment comprising the steps of applying a first composition comprising hydrogen peroxide to a human oral cavity; applying a second composition comprising a metal selected from the group consisting of copper, tin and/or zinc after said first composition; applying a third composition comprising chlorhexidine after said second composition; and applying a fourth composition comprising essential oils after said third composition, wherein each composition is applied two times per day for a period of time of about four days.

Still other aspects of the present invention may include applying a fifth composition after the fourth day, said fifth composition comprising a metal selected from the group consisting of copper, tin and/or zinc; and applying a sixth composition comprising essential oils, after said fifth composition.

Further still other aspects of the present invention may include a halitosis treatment regimen comprising gargling.

The foregoing and other features and advantages of the present invention will be apparent from the following more detailed description of the particular embodiments of the invention.

### DETAILED DESCRIPTION OF EMBODIMENTS OF THE INVENTION

As discussed above, embodiments of the present invention relate to a method of oral treatment. The method of oral treatment includes a method of oral treatment of halitosis. The methods and compositions useful in the present invention are described below.

Prior to the treatment method of the present invention, the teeth, gums, cheek, palate and the tongue may be brushed. It is preferred to brush as far back in the oral cavity as possible, which may result in gagging, for a period of time of at least about two minutes Toothpaste may be used.

The method of oral treatment to eliminate halitosis and/or other odors comprises the use of compositions which chemically neutralize the odiferous molecules. The method of oral treatment of the present invention further comprises the use of compositions which mechanically remove the causative bacteria and their food sources. The method of oral treatment of the present invention still further comprises compositions which chemically deactivate the metabolic mechanism of the causative bacteria. Additionally, the method of oral treatment of the present invention comprises compositions which inhibit the bacteria from reproducing, i.e. compositions having a bacteristatic effect and compositions which chemically kill the causative bacteria, i.e. compositions which have a bactericidal effect.

In the present invention at least two compositions, particularly at least three compositions are applied to the human oral cavity. The treatment regimen of the present invention follows a predetermined and/or sequential order.

In one embodiment, the present invention relates to applying the compositions to the oral cavity by gargling. Generally, gargling is a physical act wherein a person tilts the head back allowing a mouthful of liquid to sit in the upper throat. Air is then expelled from the lungs, causing the liquid to bubble and undulate throughout the throat and mouth region.

In the method of the present invention, gargling comprises getting a liquid onto the furthest back portion of the tongue and deep into the throat. The rear of the tongue is targeted by sticking it out and making an “aaahhh” sound. The back of the throat is reached by relaxing the tongue, letting it fall back, loosely, and making the “oooooo” sound. The two sounds may be alternated with occasional/intermittent mouth swishing when a break is needed. Gargling is not merely swishing liquid around your mouth, teeth and gums. It is believed that gargling, as described above, increases the effectiveness of the oral treatment method of the present invention.

The first composition comprises an effective amount of an agent that chemically neutralizes the odiferous molecules; mechanically removes the causative bacteria and their food sources; and has both a bacteristatic and bactericidal effect. Suitable agents include, but are not limited to, hydrogen peroxide and/or carbamide peroxide and/or chlorine dioxide. Hydrogen peroxide is preferred. The term “effective amount” as used herein means an amount of an agent sufficient to significantly induce a positive effect in the oral treatment method of the present invention. For example, hydrogen peroxide may be used in an amount in the range of from about 0.5% to about 3.0% by weight, particularly in an amount of about 3.0% by weight. It is believed that hydrogen peroxide breaks up microorganism biofilm structure in the oral cavity so the causative bacteria are more susceptible to treatment.

The second composition of the present invention comprises an effective amount of an agent that chemically deactivates the metabolic mechanism of the causative bacteria. Metals are useful in the second composition of the present invention. Suitable metals include, but are not limited to, metals selected from the group consisting of copper, tin and/or zinc. Metal salts are preferred, particularly zinc gluconate. For example, zinc gluconate may be used in an amount in the range of from about 0.001% to about 1.5% by weight, particularly in an amount in the range of from about 0.05% to about 1.0% by weight. Seed or pulp extracts may also be included such as grapefruit seed.

The third composition comprises an effective amount of a bactericidal agent. Suitable bactericidal agents include, but are not limited to, chlorhexidine and/or cetylpyridinium chloride and/or povidone-iodine. Chlorhexidine is preferred. For example, chlorhexidine gluconate may be used in an amount in the range of from about 0.025% to about 0.2% by weight, particularly in an amount of about 0.12% by weight. Cetylpyridinium chloride may be used in an amount in the range of from about 0.025% to about 0.10% by weight, particularly in an amount of about 0.07% by weight. It is believed that chlorhexidine exhibits excellent causative bacteria attachment capability.

The fourth composition comprises an effective amount of a bacteristatic and bactericidal agent. Suitable bactericidal and bacteristatic agents include, but are not limited to, essential oils and extracts. Essential oils which may be used include essential oils of peppermint, red thyme, cinnamon bark, eucalyptus globules, lavender, tea tree, spearmint, and clove and/or mixtures thereof. Extracts which may be used include eugenol, thymol, carvacrol, menthanol, eucalyptol, and totarol and/or mixtures thereof. It is believed that the essential oils penetrate the microorganism biofilm structure found in the oral cavity and kill or retard the growth of the causative bacteria.

The fifth composition, similar to the second composition, includes an agent which chemically neutralizes the odorous molecules and chemically deactivates the metabolic mechanism of the causative bacteria. Metals are useful in the fifth composition of the present invention. Suitable metals include, but are not limited to, metals selected from the group consisting of copper, tin and/or zinc. Zinc is preferred. It is believed that zinc, for example, binds to the protein receptor sites on the bacteria, suppressing the metabolic process that creates the odor causing compounds. Sodium chlorite may also be included.

Similar to the fourth composition, the sixth composition comprises an effective amount of a bacteristatic and bactericidal agent. Suitable bactericidal and bacteristatic agents include, but are not limited to, essential oils and extracts. Essential oils which may be used include essential oils of peppermint, red thyme, cinnamon bark, eucalyptus globules, lavender, tea tree, spearmint, and clove and/or mixtures thereof. Extracts which may be used include eugenol, thymol, carvacrol, menthanol, eucalyptol, and totarol. It is believed that the essential oils and extracts penetrate the microorganism biofilm structure found in the oral cavity and kill or retard the growth of the causative bacteria.

Compositions of the present invention may comprise a carrier material into which other ingredients are solubilised, dispersed or otherwise mixed. Depending upon the type of composition in question the carrier material can differ. For example, mouth wash compositions commonly have a carrier material which comprises from about 20:1 to about 2:1 aqueous alcoholic matrix.

The compositions of the present invention are prepared by standard techniques well known to those skilled in the art. Other ingredients useful in the compositions herein include, but are not limited to, anti-plaque agents, desensitizing agents, anti-calculus agents, surfactants, nutrients, sweeteners and/or flavoring agents. Typical amounts of each of the six compositions of the present invention are in the range of from ⅛ ounce to about 1 ounce. Generally about ¼ ounce of each composition is used per application.

The four steps as discussed above should be performed twice a day for a period of four days. After four days, a maintenance treatment may be performed using the fifth and sixth compositions. The maintenance treatment should be carried out twice daily after brushing. It is believed that the compositions of the present invention must reach the target area, the rear of the tongue and deep into the throat to be effective in the treatment of halitosis.

The compositions of the present invention must remain in contact with the oral cavity for an adequate amount of time to be effective. Generally, the amount of time for the initial treatment regimen of the present invention is in the range of about to about 30 seconds to 120 seconds. Preferably, the amount of contact time is about 60 seconds. Generally, the amount of time for the maintenance treatment of the present invention is in the range of from about 15 seconds to 120 seconds. Preferably, the amount of contact time is about 30 seconds. In each step of the present invention, it is preferred to reach the target area, i.e. the rear of the tongue and deep into the throat for the entire contact time.

It is believed that the compositions exert a synergistic effect and operate by different mechanisms. It is also believed that the specified application order has an effect on the halitosis treatment of the present invention.

The embodiments and examples set forth herein were presented in order to best explain the present invention and its practical applications and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims. Accordingly, any components of the present invention indicated in the drawings or herein are given as an example of possible components and not as a limitation.

What is claimed is:

1. A method for providing oral treatment comprising the steps of applying at least two compositions to a human oral cavity selected from the group consisting of a first composition comprising hydrogen peroxide; a second composition comprising a metal selected from the group consisting of copper, tin and/or zinc; a third composition comprising a bactericidal agent; and/or a fourth composition comprising essential oils.

2. The method of claim 1 wherein at least three compositions are applied to the human oral cavity.

3. The method of claim 1 wherein said first composition, second composition, third composition and fourth composition are applied in sequential order.

4. A method for providing oral treatment comprising the steps of applying a first composition comprising hydrogen peroxide to a human oral cavity; applying a second composition comprising a metal selected from the group consisting of copper, tin and/or zinc after said first composition; applying a third composition comprising a bactericidal agent after said second composition; and applying a fourth composition comprising essential oils after said third composition.

5. The method of claim 4 wherein the oral treatment is for halitosis.

6. The method of claim 4 wherein the bactericidal agent is selected from the group consisting of chlorhexidine and/or cetylpyridinium chloride.

7. The method of claim 4 wherein each composition is applied by gargling

8. The method of claim 7 wherein said gargling reaches the rear of the tongue and the throat.

9. The method of claim 8 wherein an aaahhh sound is made during said gargling

10. The method of claim 7 wherein each composition is gargled for a period of time of about 60 seconds.

11. The method of claim 4 wherein each composition is in an amount of about 0.25 ounces.

12. The method of claim 4 wherein said second composition further comprises grapefruit seed extract.

13. The method of claim 4 wherein the second composition comprises zinc.

14. The method of claim 13 wherein the zinc is in the form of zinc gluconate

15. The method of claim 4 wherein each composition is applied two times per day for a period of time of about four days.

16. The method of claim 15 further comprising applying a fifth composition after the fourth day, said fifth composition comprising a metal selected from the group consisting of copper, tin and/or zinc; and applying a sixth composition comprising essential oils, after said fifth composition.

17. The method of claim 16 wherein the fifth and sixth compositions are applied daily.

18. The method of claim 16 wherein the fifth and sixth compositions are applied by gargling

19. The method of claim 16 wherein the fifth and sixth compositions are applied for 30 seconds.

20. The method of claim 16 wherein the metal is zinc.

21. The method of claim 16 wherein the fifth composition further comprises sodium chlorite.
